(19) United States
(12) Patent Application Publication
(10) Pub. No.: US 2006/0171899 A1
(43) Pub. Date: Aug. 3, 2006

(54) Water-stabilized aerosol formulation system and method of making

(76) Inventors: Akwete Adjei, Bridgewater, NJ (US); Anthony J. Cutie, Bridgewater, NJ (US); Yaping Zhu, East Brunswick, NJ (US)

Correspondence Address: Jonathan N. Provoost;KOS Pharmaceuticals, Inc., 1 Cedar Brook Drive, Cranbury, NJ, 08512-3618, US

(21) Appl. No.: 11/331,386

(22) Filed: Jan. 11, 2006

Related U.S. Application Data
(63) Continuation-in-part of application No. 10/234,825, filed on Sep. 3, 2002.
Application No. 10/234,825 is a continuation-in-part of application No. 09/619,183, filed on Jul. 19, 2000, now abandoned.
Application No. 10/234,825 is a continuation-in-part of application No. 09/209,228, filed on Dec. 10, 1998, now Pat. No. 6,261,539.

(51) Int. Cl. A61K 48/00; A61K 38/28; A61K 38/22; A61K 38/18; A61K 38/13; A61K 38/09; A61K 9/14; A61K 31/727; A61K 39/395; A61K 31/4965; A61L 9/04

(52) U.S. Cl. 424/46; 514/2; 514/3; 514/44; 514/11; 514/15; 514/56; 424/130.1; 424/184.1; 514/43; 514/255.06

(57) ABSTRACT

The invention relates to an aerosol formulation system comprising a primary package system and an aerosol formulation therein wherein the aerosol formulation comprises insulin, a propellant and an amount of water sufficient to reach equilibrium quantities based on the moisture sorption rate diffusing across the primary package system in which the formulation is contained. In addition, the invention relates to a process for preparing the aerosol formulation systems as described herein.

This application is a continuation-in-part of U.S. patent application Ser. No. 10/234,825, filed Sep. 3, 2002, pending, which is a continuation-in-part of U.S. patent application Ser. No. 09/619,183, filed Jul. 19, 2000, abandoned, which is a continuation-in-part of U.S. patent application Ser. No. 09/209,228, filed Dec. 10, 1998, now issued as U.S. Pat. No. 6,261,539.

### FIELD OF THE INVENTION

The invention relates to an aerosol formulation system comprising a primary package system and an aerosol formulation therein containing predominantly amorphous insulin, a propellant and water. In addition, the invention relates to a process for preparing the aerosol formulation systems as described herein.

### BACKGROUND OF THE INVENTION

It is known in the art that the presence of water in conventional aerosol formulations often results in a number of potential problems, e.g., instability of the formulation, erratic dose delivery, and, in some cases, free radical reactions in the propellant. (Chengjiu Hu & Robert O. Williams III, Moisture Uptake and Its Influence on Pressurized Metered-Dose Inhalers, Pharm. Devel. and Tech. 2000 5(2), 153-162; Hugh D. C. Smyth, The influence of formulation variables on the performance of alternative propellant-driven metered dose inhalers, Advanced Drug Delivery Reviews 2003 55, 820-821). Therefore, with the exception of the small molecule crystal beclomethasone dipropionate monohydrate formulation of U.S. Pat. No. 5,695,744, persons skilled in the art have generally accepted that conventional aerosol formulations should be maintained substantially free of water. The rigorous exclusion of atmospheric moisture during both the manufacture and storage of such formulations, referred to as “developed” or “nascent” formulation water, increases the difficulties of preparing satisfactory stable aerosols containing a drug and raises the overall cost of the final product, especially when a moisture barrier, e.g. foil pouching, is included as a secondary package.

Protein and peptide drugs present a unique challenge for the formulation of stable medicaments in an aerosol formulations because of their size, structure and stability.

Further, as is known in the art, it is important that the therapeutic agent of an aerosol formulation be uniformly dispersed throughout the aerosol formulation such that the pressurized dose discharged from a metered dose valve is reproducible. Rapid creaming, settling, or flocculation, particularly of the therapeutic agent after agitation, are common sources of dose irreproducibility in suspension formulations. This is especially true where a binary aerosol formulation containing only medicament and propellant, e.g. 1,1,1,2-tetrafluoroethane, is employed. Sticking of the valve also can cause dose irreproducibility. Most notably, to date, there has been no successful commercialization of an aerosolized insulin formulation which overcomes the above-noted problems and which can effectively and efficiently deliver insulin to a patient in need thereof.

Applicants have discovered that by adding water to the solid drug formulation during the manufacture process, rather than seeking to eliminate it, applicants can obtain a stable aerosol formulation system having greatly reduced moisture ingress, thereby providing a product with comparable or improved suspension quality, dosing uniformity, content uniformity, and shelf-life then the essentially water free products of the prior art.

### SUMMARY OF THE INVENTION

The invention provides an aerosol formulation system comprising:

(a) a primary package system, and

(b) a formulation, wherein said formulation comprises (i) a protein or peptide, (ii) a propellant, and (iii) an amount of water sufficient to reach equilibrium quantities based on the moisture sorption rate diffusing across the primary package system in which the formulation is contained.

Further, the invention provides for a process for preparing an aerosol formulation system comprising:

1) forming a primary slurry comprising:
a) a protein or peptide;
b) propellant; and
c) water;

2) milling said primary slurry in one or more mills to form a final slurry; and

3) filling the final slurry into a primary package system.

An embodiment of the process of the invention provides for adding a first portion of the propellant to the primary slurry and adding a second portion of the propellant subsequent or during the milling step. Alternatively, a supplementary propellant may be added after the filling step.

The aerosol formulation systems of the present invention are useful for the systematic and/or topical application of proteins or peptides, such as insulin, in the area of the bronchi and bronchioles, and particularly, peripheral lung.

The use of added water as a stabilizing agent in the present invention provides unique benefits over other large molecule aerosol formulations because it dramatically reduces rate of moisture ingress under both normal and accelerated storage conditions. Further, the addition of water into the primary slurry facilitates the micronization of crystal insulin to predominantly amorphous insulin during the milling process and eliminates unwanted recrystalization and agglomeration. As a result, the aerosol formulation systems of the present invention demonstrate enhanced chemical and physical stability of the formulation. Where other stabilizers such as surfactants and alcohols, for example, tie up the protein or peptide particles, water permits formation of a stable, substantially amorphous structure of the API in the formulation of the present invention.

### BRIEF DESCRIPTION OF THE DRAWINGS

FIG. 1 is cross section of a typical primary package system for use in an MDI.

FIG. 2 is a graph generated using the 6 month real time data from Table 1 below to populate Equation A11, which allows one to generate an estimate for the equilibrium quantities of water (where the slope of the graph approaches zero) generated using the process of the invention.

FIG. 3 is a flow diagram an in situ manufacturing process for preparing an aerosol formulation system in accordance with an embodiment of the invention.

FIG. 4 is a series of photographs demonstrating suspension uniformity of water-stabilized MDI formulations of rh-insulin.

FIG. 5 is a chart illustrating comparative content uniformity for multiple prototype aerosol formulation systems of the current invention.

FIG. 6 is a graph illustrating the change in mean particle size as a function of temperature and time for a prototype aerosol formulation system in accordance with the present invention.

FIG. 7 illustrates log-normal distribution of cascade impact to acquire the mass median aerodynamic diameter (MMAD) and geometric standard deviation (GSD) of the mean.

FIGS. 8a and 8b illustrate a comparison of standard crystal insulin (FIG. 8a) versus the predominantly amorphous insulin contained in the aerosol formulation systems of the present invention (FIG. 8b).

FIG. 9 illustrates a measure of moisture ingress into the primary package system for a prototype aerosol formulation system in accordance with the present invention.

### DETAILED DESCRIPTION

The amount of water added to the formulation of the present invention is an amount sufficient to reach equilibrium inside and outside the primary package system based on the moisture sorption rate diffusing across the moisture permeable barriers typically contained in a primary package system, such as a pMDI. Any type of water may be used, provided it meets U.S. Pharmacopeia (USP) standards. Preferably, the water is non-carbonated.

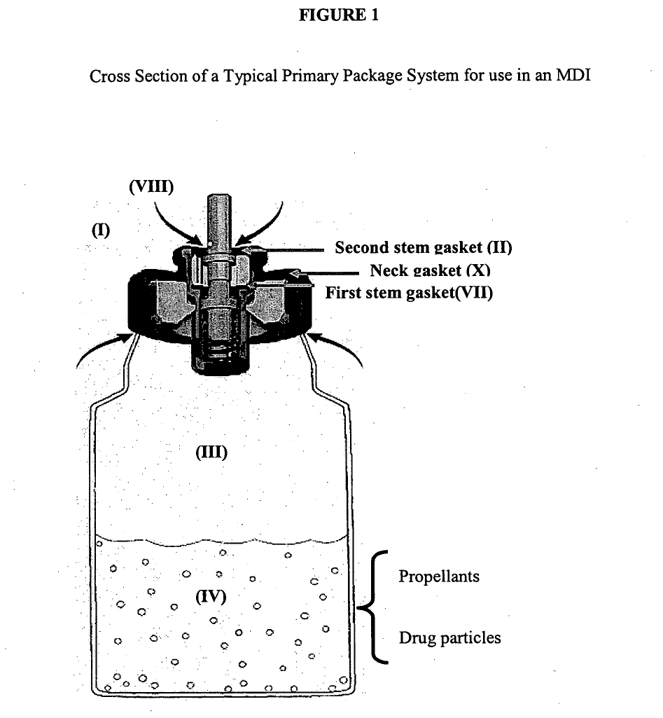

FIG. 1 illustrates a cross section of a typical pMDI package system. The package system contains a drug suspension (IV) comprising solid drug particles (IVb) suspended in a liquid propellant (IVA) or solvent, and a headspace (III), representing the interior portion of the package system containing the compressed gas or propellant vapor. Standard products may also contain solvents and/or surfactants within the drug suspension (IV). Moisture permeable barriers include common components such as the second stem gasket (II), first stem gasket (VII), actuator hole (VIII) and neck gasket (X), across which external moisture can enter the headspace over a period of time.

The amount of surplus water added to the solid drug formulation sufficient to reach equilibrium across the moisture permeable barriers of the primary package system is dependent upon the total pseudo-steady rate of moisture transfer across those permeable barriers. Further, the amount of moisture transfer is also related to the polarity of the propellant used in the formulation (i.e., the solubility of the water in the propellant). Thus, a propellant having a higher solubility of water would generally result in greater moisture ingress into the primary package system.

Although one skilled in the art may employ various means to determine the moisture transfer across a permeable barrier, one embodiment of the invention employs the following series of equations to determine the pseudo-steady rate of moisture transfer across a permeable membrane, such as the combined moisture permeable membranes of a typical pMDI.

Using Fick's Law as a guide, one can describe the pseudo-state rate (in grams per second) of moisture transfer across a thin membrane (i.e., moisture transfer into the primary package device through all permeable barriers) by the following equation:

$$\frac{ⅆ {m}_{\dot{w}}}{ⅆ t} = \frac{18.01 {D}_{w} {H}_{w} A}{δ} ( Δ {C}_{w} ) \qquad Eq . A1$$

where:
D_{w}=diffusion coefficient of water (cm² sec⁻¹)
A=surface area through which mass transfer occurs (cm²)
H_{w}=partition coefficient of water
δ=membrane thickness (cm)
ΔC=difference in diffusant conc. on each side of the membrane (mol cm⁻³)

The diffusant concentration of water (C_{w}) on each side of the membrane in terms of partial pressure (p_{w}) can be calculated by Equation A2 wherein the concentration is directly proportional to the partial pressure, assuming R and T remain constant.

$${C}_{w} = \frac{{p}_{w}}{RT} \qquad Eq . A2$$

where R is the proportionality constant (or gas constant) and T is temperature in degrees Kelvin and the proportionality constant, is parametrically dependent on gasket material and thickness, valve configuration and temperature. The permeability coefficient of water, P_{w}, has the units of mass per time. The term C_{w} may also be expressed in terms of water activity (a_{w}) as follows:

$${C}_{w} = \frac{{a}_{w} {P}_{w}^{o}}{RT} \qquad Eq . A3$$

where P_{w}^{o} is the partial pressure of water as a solvent. Applying equations A1 and A3 to both sides of the yields:

$$\frac{ⅆ M}{ⅆ t} = \frac{18.01 {D}_{w} {H}_{w} {Ap}_{w}^{o}}{δ RT} ( {a}^{in} - {a}^{out} ) \qquad Eq . A4$$

where p_{w}^{o} is the partial pressure of water at 273° Kelvin and the difference in the activity of water (Δa) is described by Δa=a^{out}−aⁱⁿ where a^{out}−aⁱⁿ represents the activity of water (a) outside and inside the canister, and the ratio of the mass of water (m_{w}) to the mass of the sample formulation (m_{f}=mass of drug, propellant and water) is represented by:

$$M = \frac{{m}_{w}}{{m}_{f}}$$

The normalized version of equation A4 is:

$$\frac{ⅆ M}{ⅆ t} = \frac{{p}_{w}}{{m}_{f}} ( {a}^{in} - {a}^{out} ) or = \frac{{P}_{w}}{{m}_{f}} Δ a \qquad Eq . A5$$

where the permeability coefficient may be described as

$${P}_{w} = \frac{18.01 {D}_{w} {H}_{w} {Ap}_{w}^{o}}{δ RT} \qquad Eq . A6$$

Equation A5 describes the proportionality between the total moisture transferred per unit time into the canister, dM/dt, and the difference in the activity of water, a, outside and inside the canister (i.e., the level of non-equilibrium inside and outside the canister).

The pseudo-steady state rate of moisture transfer across the permeable membranes of the canister is taken together with the existing moisture content present in the condensed phase of the solid drug formulation, i.e., nascent formulation water. Although one skilled in the art may use various means to determine nascent water concentration, one embodiment uses Karl Fischer titration to estimate the existing moisture content present in the condensed phase.

For example, moisture content in an insulin MDI formulation is determined by Coulometric Titration. The formulation is actuated into the titrator which contains a “single solution” Karl Fischer Reagent. The determination of water with the Karl Fisher reagent is based upon the quantitative reaction of water with iodine and an anhydrous solution of sulfur dioxide in the presence of a buffer, and the moisture result is reported in parts per million. The activity of water in the condensed phase can be written as:
a=γx Eq. A7
where x is the mole fraction and γ is the activity coefficient of water in the condensed phase. The mole fraction is defined as:

$$x = \frac{{n}_{w}}{{n}_{f }} = \frac{{n}_{w}}{{n}_{w} + {n}_{p} + {n}_{s}} \qquad Eq . A8$$

where n is the number of moles and the subscripts p and s refer to propellant and surfactant, respectively, for a formulation utilizing a surfactant quantity. The mole fraction of water in the condensed phase reduces to:
x=MΓ Eq. A9
by recognizing that nf≈n, if the moles of water and surfactant are negligible compared to the moles of propellant (n_{w}+nₛ<<nₚ). Also, the constant Γ has been used to replace the ratio of formula weights (Fₚ/F_{w}). Finally, using the above expressions for the activity and mole fraction of water, Eq. A7 can be rewritten as:

$$\frac{ⅆ M}{ⅆ t} = \frac{{P}_{w}}{{m}_{f}} Γγ M = \frac{{P}_{w}}{{m}_{f}} {a}^{out} \qquad Eq . A10$$

Since the activity of water in the environmental chamber, a^{out}, does not change, and if it is assumed that the activity coefficient is constant, the mass transfer equation can be recognized as a first order linear, non-homogenous ordinary differential equation. Moisture content as a function of time M(t) is:

$$M ( t ) = {M}_{∞} - ( {M}_{∞} - {M}_{o} ) exp - ( \frac{{P}_{w}}{{m}_{f}} Γγ t ) \qquad Eq . A11$$

where exp is the exponent, M_{∞} is the equilibrium moisture level for a specific temperature and humidity and Mₒ is the initial moisture content. Using the above linear equation, one can predict the moisture content that will enter into the canister across the permeable barriers over a period of time, t, until reaching a state of equilibrium (where the slope approaches 0).

The equation A11 can be fit with real time data to thereafter extrapolate what equilibrium quantities of water would be necessary to “spike” into the formulation initially to reach equilibrium. Estimates of equilibrium quantities are based on the amount of water needed to slow down the ingress of moisture into the canister for a reasonable period of time, e.g., three years of storage.

For example, Table 1 below illustrates a 6 month real time data acquired using a prototypical pMDI model at 25° C. RSD is relative standard deviation.

TABLE 1

| Storage Conditions | | | | | |
|---|---|---|---|---|---|
| | Temper- | Moisture Content (ppm) ± RSD | | | |
| Time | ature | Lot 1 | Lot 2 | Lot 3 | Lot 4 |
| Initial | — | 154 ± 8.2 | 152 ± 15.2 | 199 ± 7.0 | 316 ± 6.3 |
| 1 | 25° C./ | 384 ± 9.7 | 401 ± 3.3 | 422 ± 0.9 | 403 ± 3.9 |
| Month | 60% RH | | | | |
| 3 | 25° C./ | 512 ± 19.9 | 521 ± 11.1 | 595 ± 7.6 | 582 ± 3.7 |
| Months | 60% RH | | | | |
| 6 | 25° C./ | 433 ± 11.2 | NA | NA | NA |
| Months | 60% RH | | | | |

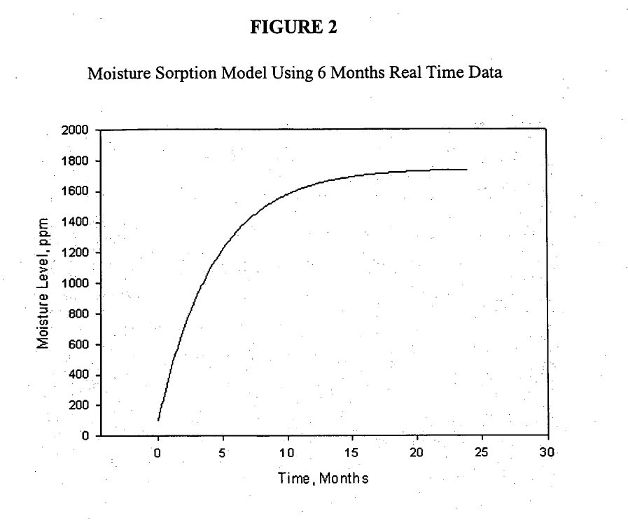

Using the 6 month real time data above to populate Equation A11, one can generate an estimate for the equilibrium quantities of water (where the slope of the graph approaches zero) as per the graphical information (FIG. 2) generated below using the process described herein.

Therefore, one skilled in the art can estimate the amount of water that will enter the canister over time in order to reach equilibrium. According to the present invention, adding this estimated amount of water into the product formulation during initial manufacture will greatly reduce, if not prevent, additional water moisture being drawn into the canister during the life of the product. In this way, applicants have found that problems normally associated with moisture seep into the canister, e.g., instability and degradation of the drug and product formulation, may be avoided by adding initially an amount of water sufficient to reach equilibrium quantities.

In certain instances where the original moisture present in the bulk drug (e.g., insulin) is of an intrinsic amount, or where water content will remain trapped into the physical structure of the protein or peptide and therefore does not ingress into the formulation, this moisture content may be of an insignificant level to impact the equilibrium kinetics to any degree of statistical significance.

A further embodiment of the invention relates to a process for preparing the inventive aerosol formulation systems described above. In it's simplest embodiment, the invention includes a process for preparing an aerosol formulation system comprising:

1) forming a primary slurry comprising:
a) a protein or peptide;
b) propellant; and
c) water;

2) milling said primary slurry in one or more mills to form a final slurry; and

3) filling the final slurry into a primary package system;

wherein said protein or peptide comprises of 0.01% to 20.00% w/w (percent weight relative to total weight of the formulation), preferably of 0.10% to 10.00% w/w, more preferably of 0.25% to 6.00% w/w of the final slurry, said propellant comprises 99.99% to 80.00% w/w, preferably of 99.90% to 90.00% w/w, more preferably of 99.75% to 94.00% w/w of the final slurry and said water comprises 0.03% to 0.20% w/w, preferably of 0.03% to 0.10% w/w, more preferably of 0.05% to 0.07% w/w of the final slurry.

As used herein, the term protein may include any protein or peptide refers to a complex, high polymer containing carbon, hydrogen, oxygen, nitrogen, and usually sulfur and composed of chains of amino acids connected by peptide linkages. A peptide or polypeptide (or oligopeptide) as use herein refers to a class of compounds of acid units chemically pound together with amide linkages (—CONH—) with elimination of water. Examples of proteins or peptides include those having a molecular size ranging from 0.5 K Dalton to 150 K Dalton, such as, but not limited to insulin, insulin analogs, amylin, glucagon; immunomodulating peptides, interleukins, erythropoetins, thrombolytics, heparin; anti-proteases, antitrypsins, amiloride, rhDNase, antibiotics, other antiinfectives, parathyroid hormones, LH-RH and GnRH analogs, nucleic acids, DDAVP, calcitonins, cyclosporine, ribavirin, hematopoietic factors, cyclosporine, vaccines, immunoglobulins, vasoactive peptides, antisense agents, genes, oligonucleotide. In addition, a protein or peptide may include pharmaceutically acceptable salts and solvates of the proteins or peptides, as described above and hereinafter.

Preferably, the protein or peptide is insulin and said insulin is micronized during the milling process step to form a predominantly amorphous insulin wherein the amorphous insulin has a volumetric mean particle size (VMPS) of 1 μm to 25 μm and/or Mass Median Aerodynamic Diameter (MMAD) of 1 μm to 15 μm, preferably the volumetric mean particle size is in the range of 1 μm to 15 μm and/or MMAD in the range of 1 μm to 10 μm, more preferably volumetric mean particle size in the range of 1 μm to 5 μm and/or MMAD in the range of 1 μm to 5 μm.

The term “insulin” shall be interpreted to encompass insulin analogs, natural extracted human insulin, recombinantly produced human insulin, insulin extracted from bovine and/or porcine sources, recombinantly produced porcine and bovine insulin and mixtures of any of these insulin products. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution. Synthetically produced insulin can be made according to any known process. In a preferred embodiment, rh-insulin (recombinant human insulin) is employed.

The term “recombinant” refers to any type of cloned biotherapeutic expressed in procaryotic cells or a genetically engineered molecule, or combinatorial library of molecules which may be further processed into another state to form a second combinatorial library, especially molecules that contain protecting groups which enhance the physicochemical, pharmacological, and clinical safety of the biotherapeutic agent.

A further embodiment includes a process comprising:
1) forming a primary slurry comprising:

a) insulin;

b) a first portion of propellant; and

c) water;
2) milling said primary slurry to form predominantly amorphous insulin;
3) adding a second portion of said propellant to the milled slurry to form a final slurry; and
4) filling the final slurry into a primary package system.

Preferably, the first portion of the total propellant is in the range of about 64.0% w/w to about 80.0% w/w, preferably about 72.0% w/w to about 79.92% w/w, more preferably about 75.2% w/w to about 79.8% w/w, and the second portion of the propellant is in the range of about 16.0% w/w to about 20.0% w/w, preferably about 18.0% w/w to about 19.98% w/w, more preferably about 18.80% w/w to about 19.95% w/w.

An additional embodiment includes forming a “pre-mix” of propellant and water prior to forming the primary slurry of step 1, such that the process comprises:
1) forming a primary slurry comprising:

a) insulin;

b) a first portion of propellant; and

c) a pre-mix of water and propellant;
2) milling said primary slurry to form predominantly amorphous insulin;
3) adding a second portion of said propellant to the milled slurry to form a final slurry; and
4) filling the final slurry into a primary package system.

In the embodiment enumerated above, the pre-mix of water and propellant is formed using conventional means known to those skilled in the art and is preferably mixed adequately prior to addition to form the primary slurry. When forming a pre-mix, the proportion of propellant in the pre-mix is in the range of 50.00% to 40.00% w/w, preferably of 49.95% to 45.00% w/w, more preferably of 49.88% to 47.00% w/w, and the first portion of the propellant is in the range of 30.00% to 24.00% w/w, preferably of 29.97% to 27.00% w/w, more preferably of 29.93% to 28.2% w/w, and the second portion of the propellant is in the range of 20.00% to 16.00% w/w, preferably of 19.98% to 18.00% w/w, more preferably of 19.95% to 18.80% w/w.

A further embodiment comprises adding a supplemental amount of propellant into the primary package system after filling the final slurry into the primary package system. Preferably, said supplemental amount of propellant is in the range of 0.1 to 10 times of the fill weight of the final slurry, preferably of 0.1 to 5 times of the fill weight of the final slurry, more preferably of 0.5 to 3 times of the fill weight of the final slurry.

The slurry of step one may be mixed in a pressure vessel or tank. Any suitable pressure vessel capable of withstanding the pressure of the propellant and can be appropriately fitted with an inlet and outlet valve assembly, agitation means and/or entry funnel can be used for purposes of the present invention. The various critical pressures and temperatures for the individual propellants are well known by one skilled in the art. A jacketed stainless steel tank is preferred.

The mixing and milling of the primary slurry may occur separately or in the same vessel. Where milling occurs outside the mixing vessel, more than one mixing vessel may be employed, such that the primary slurry may be circulated between two tanks through one or more mills until the insulin is micronized (i.e., the conversion of crystal insulin to a predominantly amorphous form) to a desired volumetric mean particle size. Although specific examples are provided herein, alternative variations for mixing and milling the primary slurry may be known to those skilled in the art to achieve the desired mean particle size and mixed primary slurry. As used herein, the term “amorphous” means a product, lacking distinct crystalline structure, e.g., having no molecular lattice structure that is characteristic of the solid crystal state, such as the formulation of repeating regular 3-dimensional arrangement of atoms or molecules. Amorphous includes non-crystal solid materials. “Predominantly” amorphous insulin, as used herein is insulin that is 80% to 100% amorphous, preferably 90% to 100% amorphous, more preferably 95% to 100% amorphous, or more preferably 99% to 100% amorphous.

In this way, via the milling process, one converts bulk crystalline insulin into a predominantly amorphous, energetically stabilized form during the micronization process. As a result, the package formulation system of the present invention demonstrates reduced susceptibility to the physical instabilities of aggregation, precipitation and absorption, and thereby demonstrates highly desirable levels of stability and dispersion quality.

Volumetric particle size may be measured by means known to those skilled in the art, such as, for example using an AersoSizer™. Measurement samples may be taken (manually or automated) after each pass through the mill or mills, or at any point suitable to accurately measure volumetric particle size. Morphology, texture and type of bulk drug (e.g., insulin) may influence circulation time and desired volumetric mean. Samples may be taken as often as needed, for example, as often as the completion of each pass, to determine when the desired particle size has been achieved.

Where a second tank is employed, the second tank is typically of the same type as the first. A jacketed stainless steel tank is preferred, however it will be clear to one of ordinary skill in the art that any tank suitable to the formulations contemplated in the present invention, and their methods of making, may be used. Any number of tanks and mills may be used based on manufacturing efficiency and cost of operation. Additional mills may be added to decrease total milling time or for large-scale production.

Milling may be performed using any commercially available apparatus provided the mill contains a grinding media suitable for micronizing the bulk drug (e.g., insulin). The grinding media preferably consists of hardened, lead-free glass beads, or zirconium, ceramic, or polymeric beads having a diameter of about 0.25 mm to about 1 mm. Grinding or micronization is affected by impact between the solid drug particles and the grinding media that are constantly stirred by a horizontal agitator. Preferably the mill is jacketed and has a 0.2-liter or greater capacity, and can accommodate at least 100 to 2500 ml of grinding media. Alternatively, a colloid mill may be used.

Slurry circulation rate can be controlled using appropriate flow control valves and pumps. Preferably, the slurry is circulated at a rate of about 10 to 2000 g/min, preferably 100 to 1000 g/min, most preferably 600 to 800 g/min. Additionally, the micronization step is preferably conducted at a temperature ranging from about 15° C. to about −50° C., more preferably at 5° C. to −15° C. Heat generated by the slurry during milling and heat from the environment are preferably removed by circulating a coolant through the mill and vessel jackets. Further, the micronization step is preferably conducted at a pressure ranging from about 15 pounds per square inch gravity (psig) to about 50 psig, depending on the propellant used. Pressure may be controlled by a pressure valve.

Subsequent to milling the primary slurry as described above, a second portion of propellant is added to the milled slurry to form a final slurry using means known to those skilled in the art. The final slurry is then filled into a primary package system, suitable for delivery of the final slurry formulation to form an aerosol formulation system.

The term primary package system as used herein includes aerosol canisters suitable for use in any pulmonary drug delivery system capable of dispensing a drug formulation (e.g., an insulin formulation) into the airways of a human patient for the purpose of systematic and/or topical administration of the active drug ingredient inside the lung cavity. Examples of such pulmonary drug delivery systems are metered dose inhalers (MDIs).

Preferably, the canister is a canister suitable for use as a MDI, such as lined aluminum canisters. Any suitable type of conventional aerosol canister however, may be employed, such as glass, stainless steel, polyethylene terephthalate, which are coated or uncoated, and it will be understood by those skilled in the art that the type of canister and type of coating, if any, is dependent on the particular propellant and drug used in the formulation. Aerosol canisters, as used in the present invention are generally equipped with conventional valves, such as metered dose and continuous valves, that can be used to deliver the formulations as described herein. The selection of appropriate valve assemblies for use with aerosol formulations is dependent on the particular propellant and drug being used.

Filling of the primary package system is accomplished using any equipment suitable to deliver a fixed volume of slurry and/or propellant to a canister, e.g., equipment with one or more pneumatically actuated valves to control filling weight to within appropriate specifications. Examples of suitable equipment include for example a Pamasol Double Diaphragm Pump, Pamasol Suspension Filler and Pamasol Propellant Filler (manufactured by Pamasol Willi Mader AG/DH Industries). Suitable canisters preferably range in capacity from about 10 mL to about 30 mL, more preferably from about 14 mL to about 20 mL.

Prior to filling the canisters, the canisters are typically “crimped”, i.e. sealed to maintain the formulation inside the canister. Crimping may be performed using any suitable equipment known in the art, such as a Pamasol Vacuum Crimper and may be accomplished after optional propellant purge of the canister, vacuum application to the canister, or inert gas purge of the canister in order to render the canister virtually air free. Crimping parameters can be readily determined by one of ordinary skill in the art and depend on a number of factors including canister specifications.

Optionally, an additional amount of propellant may be added subsequent to filling the canisters as described above. This additional propellant may be added into the canister through the valve of the canister to achieve the desired target weight of the canister. Further, as discussed herein, a pre-mix of water and propellant may first be formed prior to forming the primary slurry.

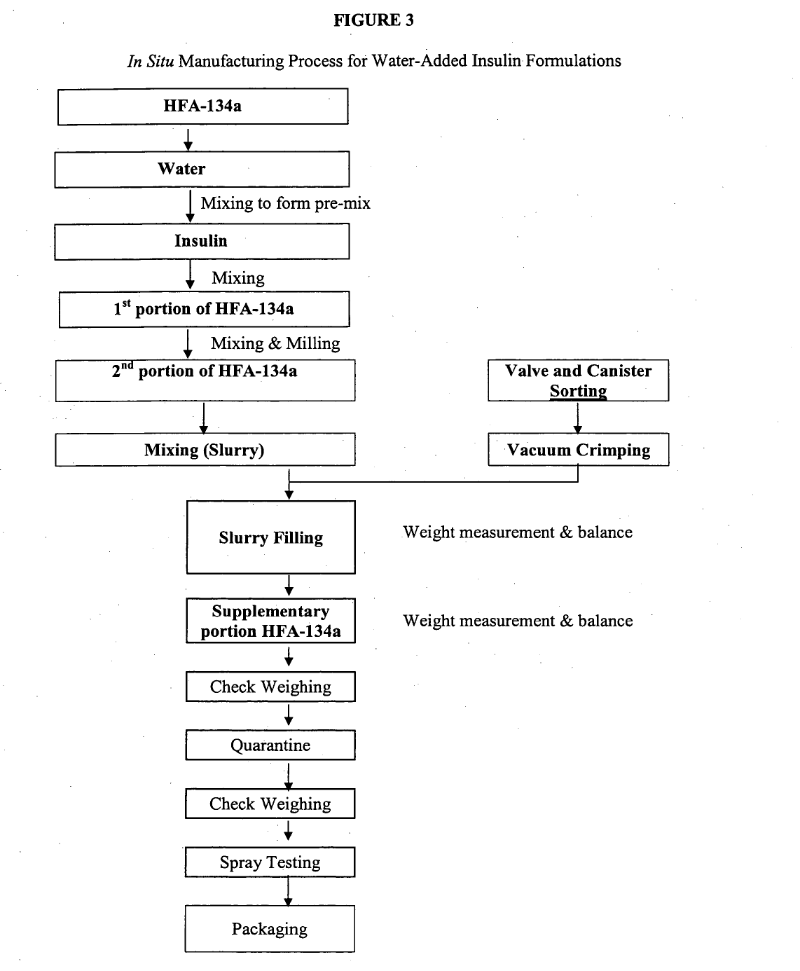

FIG. 3 is a flow diagram illustrating an in situ manufacturing process for preparing an aerosol formulation system in accordance with an embodiment of the invention. In accordance with such embodiment the processing vessels and mills are first cooled to about −15° C. Propellant, HFA-134a, is first added into the cold vessel, followed by a suitable amount of purified water, which are then mixed to form a pre-mix of propellant and water. Thereafter, into the same vessel is added insulin and a first portion of HFA-134a to form a primary slurry. The primary slurry is mixed and milled until obtaining the desired mean particle size for the insulin, thereby obtaining a slurry containing predominantly amorphous insulin. Thereafter, a second portion of HFA-134a is added to the milled primary slurry to rinse the processing equipment and form a final slurry having a desired insulin concentration. Subsequently the final slurry is transferred to an aerosol (MDI) filler and the canister is filled with a portion of the final slurry to a target weight through the valve into the canister. The filling step may include up to 5 days hold time. After holding, a supplementary portion of HFA-134a is added through the valve to achieve final target weight.

The following examples serve to better illustrate, but not limit, multiple embodiments of the invention.

### EXAMPLE 1

A closed line system containing tanks and mills having a chiller temperature set at −15° C. was set up in accordance with the process of the invention. A portion of a 6.448 kg amount of HFA-134a propellant was placed into a 1-gallon disperser tank via a bead mill containing 475 ml of cleansed glass beads and 3.25 g of stabilizing water added to the chamber of the mill. While circulating the liquid from the bead mill to the disperser tank, 48.75 g rh-insulin was introduced to the vessel using a charging funnel. Immediately thereafter, the balance of 6.448 kg of the propellant was flushed through the charging funnel into the disperser tank. Recirculation through the bead mill was initiated and continued until a mean particle diameter of about 3.5 micrometers was obtained. About 6.5 g of suspension was filled into crimped canisters and checked for leaks. Canisters were monitored to investigate the stability performance of the product. The resulting formulation contained 8.9 U rh-insulin/spray and 1027 ppm Total Water (“Total Water”=nascent water and the added stabilizing water).

### EXAMPLE 2

A closed line system containing tanks and mills having a chiller temperature set at −15° C. was set up in accordance with the process of the invention. A portion of a 3.436 kg amount of HFA-134a propellant was placed into a 1-gallon disperser tank via a bead mill containing 475 ml of cleansed glass beads and 3.46 g of stabilizing water added to the chamber of the mill. While circulating the liquid from the bead mill to the disperser tank, 51.96 g rh-insulin was introduced to the vessel using a charging funnel. Immediately thereafter, the balance of 3.436 kg of the propellant was flushed through the charging funnel into the disperser tank. Recirculation through the bead mill was initiated and continued until particle size results were obtained, a mean particle diameter 2.6 micrometers. About 5.5 g of suspension was filled into crimped canisters and checked for leak proofness. Canisters were monitored to investigate the stability performance of the product. The resulting formulation was about 8 U rh-insulin/spray and 759.9 ppm Total Water.

### EXAMPLE 3

A closed line system containing tanks and mills having a chiller temperature set at −15° C. was set up in accordance with the process of the invention. A 7.8 kg amount of HFA-134a propellant was placed into a 1-gallon disperser tank via a bead mill containing 475 ml of cleansed glass beads and 13.706 g of stabilizing water added to the chamber of the mill. While circulating the liquid from the bead mill to the disperser tank, 407.4 g rh-insulin was introduced to the vessel using a charging funnel. Immediately thereafter, 4.7 kg of the propellant was flushed through the charging funnel into the disperser tank. Recirculation through the bead mill was initiated and continued for 9 passes, following which the contents of the mill and the second vessel were flushed into the disperser tank with another 3.8 kg propellant while mixing. The final slurry concentration for the batch was 685 U/g slurry material. Varying amounts of slurry were filled into canisters that were then subsequently charged with enough propellant to yield 10 g of final aerosol product with varying concentrations of rh-insulin as follows:

2.36 g slurry+7.64 g propellant yielded 10U/spray and 842 ppm Total Water

4.51 g slurry+5.49 g propellant yielded 20U/spray and 865 ppm Total Water

7.96 g slurry+2.04 g propellant yielded 35U/spray and 1409 ppm Total Water

After 36 months of monitoring stability performance, the aerosol formulation systems of Examples 1 to 3 demonstrated little or no level of moisture ingress, nor unwanted recrystalization and agglomeration of the insulin.

### EXAMPLE 4

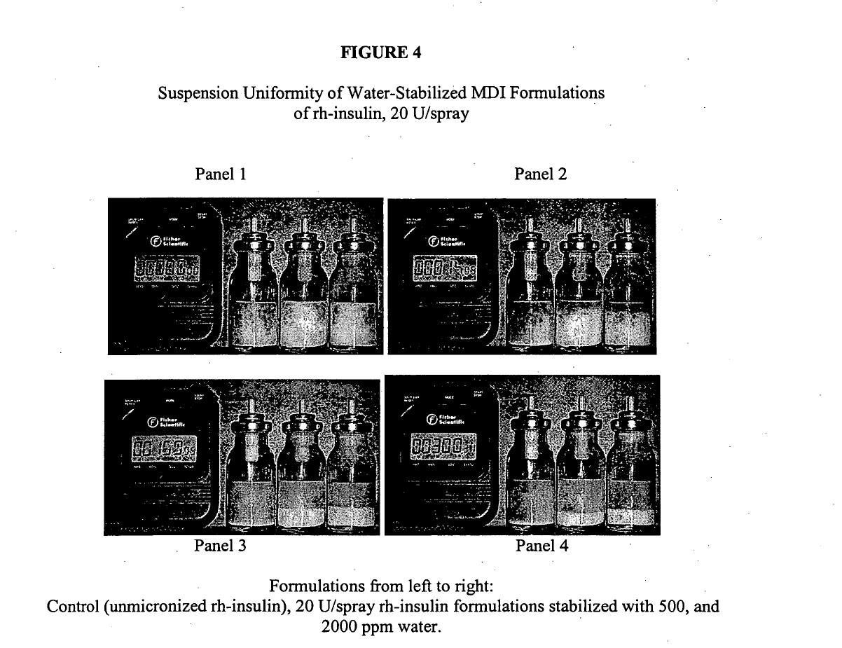

FIG. 4 illustrates a comparative study of suspension uniformity for the aerosol formulations of the invention in comparison with a sample with no water added and unmicronized rh-insulin. Three comparative prototype samples were prepared that included (from left to right in Panels 1 to 4) (i) a control containing no added water (˜0 ppm) and unmicronized rh-insulin, (ii) a 20 U/spray formulation prepared in accordance with the present invention and containing 500 ppm water and micronized rh-insulin, and (iii) a 20 U/spray formulation prepared in accordance with the present invention and containing 2000 ppm water and micronized rh-insulin. The samples were hand-shaken to sufficiently disperse the insulin particles contained therein and then placed down on a lab bench immediately thereafter as illustrated in Panel 1. Photos were taken at time intervals of 15 seconds, one minute and three minutes after shaking with visual observation of setting and floccule suspension.

As illustrated in the Photos, the prototype aerosol formulation systems of the present invention demonstrate superior suspension qualities then the control. Looking at Panel 2, one can see almost complete separation and the formation of a precipitate on the bottom of the control formulation after only 15 seconds. As a result, after only 15 seconds the control formation would not easily reconstitute uniformly upon shaking. In contrast, the aerosol formulation systems of the current invention onto completely separate, but rather exist in loosely held flocs or floccules with reduced separation and settling. As a result, minimal shaking of the aerosol formulation systems of the invention would result in uniform dispersion of the product in the suspension, thus resulting in a more predictable and dependable dose uniformity profile. Superior results compared to the control, and desirable levels of separation and settling are demonstrated by the aerosol formulation systems of the present invention of at all time intervals up to 3 minutes. As dose uniformity is dependent upon suspension quality, the stable aerosol formulation systems of the current invention evidence an ability to provide good dispersion uniformity for a longer period of time and with minimal shaking between puffs when used in an MDI.

### EXAMPLE 5

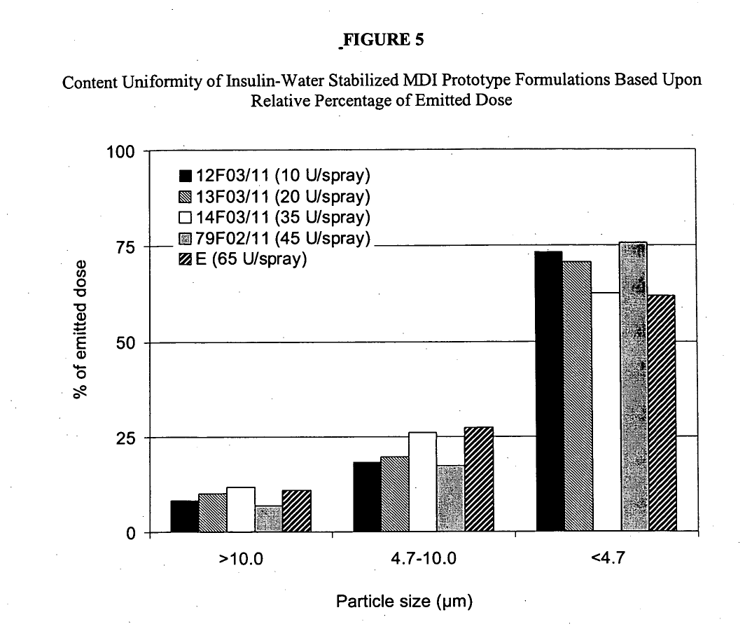

FIG. 5 illustrates comparative particle size of insulin, as a percent of emitted dose, from multiple prototype aerosol formulation systems of the current invention. The particle size of insulin was measured using an Andersen Cascade Impactor (Mark II), comprising of a vacuum pump to generate an air flow, eight stages with collection plates and a top stage with an inlet cone and induction port. When air was drawn at 28.3 Liters/minute into the cascade impactor, multiple jets of air in each stage directed insulin particles onto the surface of the collection plates for each appropriate particle size range. Insulin particles deposited on each plate were subsequently analyzed by an HPLC method. The results are plotted in FIG. 5, indicating uniform/consistent size distribution of insulin particles across the formulation strengths 10, 20, 35, 45 and 65 U/spray of the current invention.

A drug (e.g., insulin) particle size of 1.0 μm to 4.7 μm is generally preferred, as it is known in the art that drug particles of this desired size most adequately travel to and deposit in the lungs of the user, thereby providing the best delivery of the active drug ingredient as intended. Drug particles greater than 4.7 μm, even more so drug particles greater than 10.0 μm, tend to deposit in the throat or are swallowed, thereby never reaching the lung. In addition, larger drug particles can stick to the valve and canister, diminishing the amount of drug delivered per dose to the patient. Particles below 1.0 μm tend to be “exhaled” by the user in a manner similar to cigarette smoke. Further, increased levels of active drug in aerosol formulations are known to lead to increased particle size. This is primarily due to an increase in active drug particle interactions. The greater the concentration of active drug per dose or “puff”, the greater number of drug particles in the puff and the more likely the drug particle administered to the patient will be larger than desired. Thus, as the concentration of active ingredient increases, the % concentration of particles in the desired 1.0 μm to 4.7 μm range should decrease.

In contrast, the aerosol formulation systems of the current invention demonstrate the ability to provide no statistically significant difference in particle size across all measured concentration ranges. As illustrated in FIG. 5, the increase in insulin concentration for the tested prototypes does not significantly impact the amount of deliverable particle size within the desired 1.0 μm to 4.7 μm size range. Rather, the size distribution profiles of the formulation at varying active drug concentration remain relatively constant from 10 Units (U) of drug per spray to approximately 65 U/spray, illustrating that increasing drug concentration in the aerosol formulation systems of the present invention does not have an adverse effect on the desired particle size of insulin particles contained in the formulation. Rather, the percentage of emitted dose comprising certain drug particle size remains relatively constant at varying concentrations. This illustrates desirable and respirable dose proportionality of the aerosol formulation systems of the present invention at varying concentrations.

### EXAMPLE 6

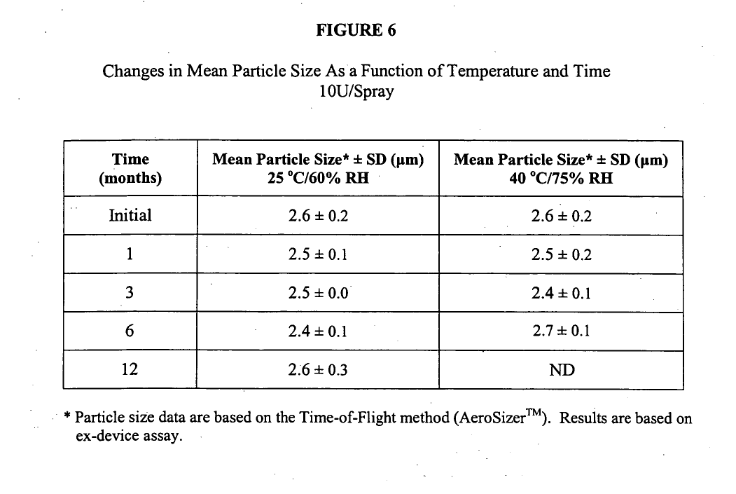

FIG. 6 illustrates the change in mean particle size as a function of temperature and time for a prototype 10U/Spray aerosol formulation system in accordance with the present invention. Particle size data was taken at 1, 3, 6 and 12 months period using the Time-of-Flight method (AeroSizer™). Results were based on ex-devise assays. As illustrated in FIG. 6 the particle size of the 10 U/spray prototype remained constant at approximately 2.6 μm through 12 months at room temperature (25° C./60% relative humidity) and through 6 months under stress (40° C./75% relative humidity).

In general, particle size of the drug product will increase as moisture enters the canister and the rate of moisture entry into the canisters of the prior art aerosol systems is typically a function of temperature and time. In contrast, the data illustrated in FIG. 6 demonstrates that the aerosol formulations of the present invention demonstrate little mean particle size increase over time, thereby evidencing no significant moisture entry into the primary package system (i.e., canister) over an extended period of time.

### EXAMPLE 7

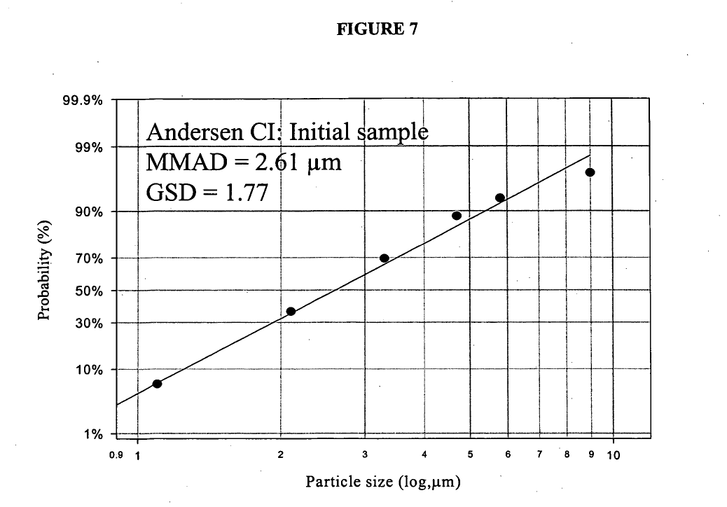

FIG. 7 illustrates log-normal distribution of cascade impact to acquire the mass median aerodynamic diameter (MMAD) and geometric standard deviation (GSD) of the mean at variable times during 12 month monitoring at room temperature using the same samples as prepared in Example 6. The size distribution of the particles in the exposure chamber was initially determined with an Andersen cascade impactor (Model Mark II, Mfr: Andersen Corporation). (see for example Physical Tests and Determinations: Aerodynamic Size Distribution, The United States Pharmacopeia, Jan. 1, 2005, pp 2364-2367). A cumulative version of the lognormal curve or more frequently, a linearized version of the cumulative curve called a “log probability plot,” is used as a surrogate for the lognormal curve. (see for example Theil C G, Cascase impactor data and the lognormal distribution: nonlinear regression for a better fit. J. Aerosol Med. 2002 Winter: 15(4), 369-378)

As is known to those skilled in the art, a particle-size mass distribution of the mass median aerodynamic diameter of 1 to 5 μm is most desired and a geometric standard Deviation should ideally be in the range of 1.5 to 3.0. With respect to the samples prepared in accordance with the invention, the calculated MMAD of particles was 2.61 μm and the GSD was 1.77.

### EXAMPLE 8

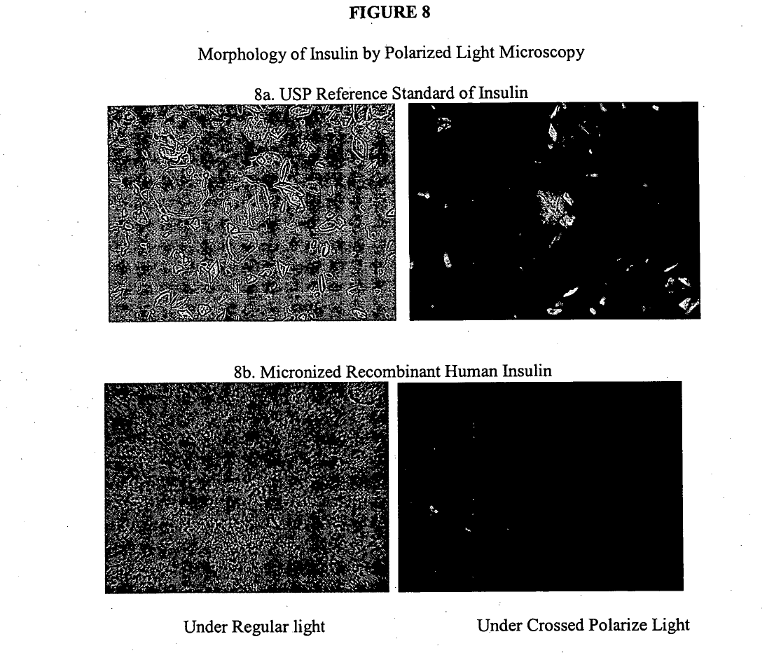

FIGS. 8a and 8b illustrate a comparison of standard crystal insulin (FIG. 8a) versus the predominantly amorphous insulin contained in the aerosol formulation systems of the present invention (FIG. 8b). The photomicrographs in FIGS. 8a and 8b were taken using an Olympus BH-2 polarizing microscope. Each sample was prepared using immersion oil with a refractive index of 1.51 and examined under polarized light. Photomicrographs 8a illustrate crystalline insulin, USP, under both regular and cross polarized light. In contrast to the crystalline structure of standard insulin, the predominantly amorphous insulin of the present invention, illustrated in the photomicrographs of 8b, demonstrate little or no polarization.

### EXAMPLE 9

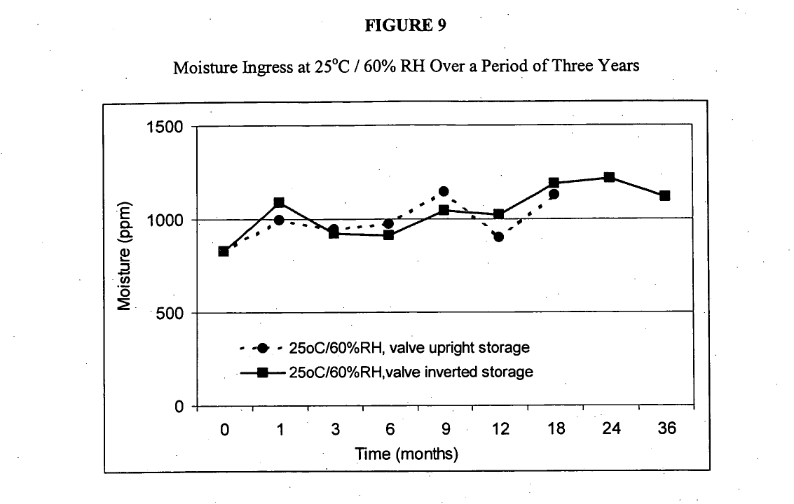

FIG. 9 illustrates a measure of moisture ingress into the primary package system (i.e., canister) for a prototype aerosol formulation system in accordance with the present invention. Samples were stored at 25° C./60% RH (relative humidity) for a period of three years with moisture levels measured at regular intervals (e.g., every three months). Moisture was measured using Karl Fischer titration, as described previously herein. The data demonstrates that the moisture content inside the canister remains constant over time, thus evidencing that moisture ingress is greatly reduced or eliminated for prolonged periods of time with the aerosol formulation systems of the invention.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entirety.

What is claimed is:

1. An aerosol formulation system comprising:
(a) a primary package system, and
(b) a formulation, wherein said formulation comprises (i) a protein or peptide, (ii) a propellant, and (iii) an amount of water sufficient to reach equilibrium quantities based on the moisture sorption rate diffusing across the primary package system in which the formulation is contained.

2. The aerosol formulation system of claim 1 wherein said medicament is selected from the group consisting of insulin, insulin analogs, amylin, glucagon; immunomodulating peptides, interleukins, erythropoetins, thrombolytics, heparin; anti-proteases, antitrypsins, amiloride, rhDNase, antibiotics, other antiinfectives, parathyroid hormones, LH-RH and GnRH analogs, nucleic acids, DDAVP, calcitonins, cyclosporine, ribavirin, hematopoietic factors, cyclosporine, vaccines, immunoglobulins, vasoactive peptides, antisense agents, genes, oligonucleotide and pharmaceutically acceptable salts and solvates thereof, and mixtures thereof.

3. The aerosol formulation system of claim 1 wherein said protein or peptide is insulin.

4. The aerosol formulation system of claim 3 wherein said insulin is predominantly amorphous insulin.

5. The aerosol formulation system of claim 4 wherein said predominantly amorphous insulin has a mass median aerodynamic diameter of about 1 μm to 15 μm.

6. The aerosol formulation system of claim 5 wherein said predominantly amorphous insulin has a mass median aerodynamic diameter of about 1 μm to 10 μm.

7. The aerosol formulation system of claim 6 wherein said predominantly amorphous insulin has a mass median aerodynamic diameter of about 1 μm to 5 μm.

8. The aerosol formulation system of claim 1 wherein said propellant is selected from the group consisting of 1,1,1,2-tetrafluoroethane and 1,1,1,2,3,3,3-heptafluoropropane, or a mixture thereof.

9. The aerosol formulation system of claim 1 wherein said water is present in an amount in the range of about 0.03% w/w to about 0.20% w/w.

10. The aerosol formulation system of claim 9 wherein said water is present in an amount in the range of about 0.03% w/w to about 0.10% w/w.

11. The aerosol formulation system of claim 10 wherein said water is present in an amount in the range of about 0.05% w/w to about 0.07% w/w.

12. The aerosol formulation system of claim 1 wherein said propellant is present in an amount in the range of about 80.0% w/w to about 99.99% w/w.

13. The aerosol formulation system of claim 12 wherein said propellant is present in an amount in the range of about 90.0% w/w to about 99.90% w/w.

14. The aerosol formulation system of claim 13 wherein said propellant is present in an amount in the range of about 94.0% w/w to about 99.75% w/w.

15. The aerosol formulation system of claim 1 wherein said protein or peptide is present in an amount in the range of about 0.01% w/w to about 20.0% w/w.

16. The aerosol formulation system of claim 4 wherein said predominantly amorphous insulin is present in an amount in the range of about 0.1% w/w to about 10.0% w/w.

17. The aerosol formulation system of claim 16 wherein said predominantly amorphous insulin is present in an amount in the range of about 0.25% w/w to about 6.0% w/w.

18. The aerosol formulation system of claim 1 wherein said primary package system is an aerosol canister.

19. The aerosol formulation system of claim 18 wherein said aerosol canister is equipped with a metered dose valve.

20. The aerosol formulation system of claim 3 comprising a formulation substantially similar to a formulation selected from the group consisting of

| | A | B | C | D | E |
|---|---|---|---|---|---|
| | (w/w %) | (w/w %) | (w/w %) | (w/w %) | (w/w %) |
| Insulin | 0.75 | 0.55 | 1.05 | 1.85 | 2.73 |
| Propellant | 99.20 | 99.43 | 98.91 | 98.08 | 97.22 |
| Water | 0.05 | 0.02 | 0.04 | 0.07 | 0.05 |

21. A process for preparing an aerosol formulation system comprising:
1) forming a primary slurry comprising:
a) a protein or peptide;
b) propellant; and
c) water;
2) milling said primary slurry in one or more mills to form a final slurry; and
3) filling the final slurry into a primary package system.

22. The process of claim 21 wherein said protein or peptide is selected from the group consisting of insulin, insulin analogs, amylin, glucagon; immunomodulating peptides, interleukins, erythropoetins, thrombolytics, heparin; anti-proteases, antitrypsins, amiloride, rhDNase, antibiotics, other antiinfectives, parathyroid hormones, LH-RH and GnRH analogs, nucleic acids, DDAVP, calcitonins, cyclosporine, ribavirin, hematopoietic factors, cyclosporine, vaccines, immunoglobulins, vasoactive peptides, antisense agents, genes, oligonucleotide and pharmaceutically acceptable salts and solvates thereof, and mixtures thereof.

23. The process of claim 22 wherein said protein or peptide is insulin and said insulin is converted to predominantly amorphous insulin during said milling step.

24. The process of claim 23 wherein said predominantly amorphous insulin has a mass median aerodynamic diameter of about 1 μm to about 15 μm.

25. The process of claim 24 wherein said predominantly amorphous insulin has mass median aerodynamic diameter of about 1 μm to about 10 μm.

26. The process of claim 25 wherein said predominantly amorphous insulin has a mass median aerodynamic diameter of about 1 μm to about 5 μm.

27. The process of claim 21 wherein said propellant is selected from the group consisting of 1,1,1,2-tetrafluoroethane and 1,1,1,2,3,3,3-heptafluoropropane, or a mixture thereof.

28. The process of claim 21 wherein said water is present in an amount in the range of about 0.03% w/w to about 0.20% w/w.

29. The process of claim 21 wherein said propellant is present in an amount in the range of about 80.0% w/w to about 99.99% w/w.

30. The process of claim 21 wherein said protein or peptide is present in an amount in the range of about 0.01% w/w to about 20.0% w/w.

31. The process of claim 23 wherein said predominantly amorphous insulin is present in an amount in the range of 0.1% w/w to about 10.0% w/w.

32. The process of claim 31 wherein said predominantly amorphous insulin is present in an amount in the range of 0.25% w/w to about 6.0% w/w.

33. A process for preparing an aerosol formulation system comprising
1) forming a primary slurry comprising:
a) insulin;
b) a first portion of propellant; and
c) water;
2) milling said primary slurry to form predominantly amorphous insulin;
3) adding a second portion of said propellant to the milled slurry to form a final slurry; and
4) filling the final slurry into a primary package system.

34. The process of claim 33 wherein said first portion of propellant is in the range of 64.0% to 80.0% w/w and said second portion of total propellant is in the range of 16.0% to 20.0% w/w.

35. The process of claim 34 wherein said first portion of propellant is in the range of 72.0% to 79.92% w/w and said second portion of total propellant is in the range of 10.0% to 19.98% w/w.

36. The process of claim 35 wherein said first portion of propellant is in the range of 75.2% to 79.8% w/w and said second portion of total propellant is in the range of 18.8% to 19.95% w/w.

37. The process of claim 33 wherein said insulin is present in an amount of about 0.01% w/w to about 20.00% w/w and said water is present in an amount of about 0.03% w/w to about 0.2% w/w

38. The process of claim 33 further comprising adding supplementary propellant into the primary package system subsequent to filling the final slurry into the primary package system.

39. The process of claim 38 wherein said supplementary propellant is in the range of about 0.1 to about 10.0 times the fill weight of the final slurry.

40. A process for preparing an aerosol formulation system comprising
1) forming a primary slurry comprising:
a) insulin;
b) a first portion of propellant; and
c) a pre-mix of water and propellant;
2) milling said primary slurry to form predominantly amorphous insulin;
3) adding a second portion of said propellant to the milled slurry to form a final slurry; and
4) filling the final slurry into a primary package system.

41. The process of claim 40 wherein the proportion of propellant in the pre-mix in the range of about 40.0% w/w to about 50.0% w/w, said first portion of propellant is in the range of about 24.0% w/w to about 30.0% w/w, and said second portion of propellant is in the range of about 16.0% w/w to about 20.0% w/w.

42. The process of claim 41 wherein the proportion of propellant in the pre-mix in the range of about 45.0% w/w to about 49.95% w/w, said first portion of propellant is in the range of about 27.0% w/w to about 29.97% w/w, and said second portion of propellant is in the range of about 18.0% w/w to about 19.98% w/w.

43. The process of claim 42 wherein the proportion of propellant in the pre-mix in the range of about 47.0% w/w to about 49.88% w/w, said first portion of propellant is in the range of about 28.2% w/w to about 29.93% w/w, and said second portion of propellant is in the range of about 18.80% w/w to about 19.95% w/w.

44. The process of claim 40 wherein said insulin is present in an amount of about 0.01% w/w to about 20.00% w/w and said water is present in an amount of about 0.03% w/w to about 0.2% w/w.

45. A method for reducing the moisture ingress into an aerosol formulation system comprising filling a primary package system with a formulation comprising:
1) a protein or peptide,
2) a propellant, and
3) an amount of water sufficient to reach equilibrium quantities based on the moisture sorption rate diffusing across the primary package system in which the formulation is contained.

46. The method of claim 45 wherein said protein or peptide is insulin.

47. The method of claim 46 wherein said insulin is predominantly amorphous insulin.
